(19) United States
(12) Patent Application Publication
(10) Pub. No.: US 2006/0167244 A1
(43) Pub. Date: Jul. 27, 2006

(54) Polymerisation inhibitor

(76) Inventors: Emyr Philips, Tingley (GB); Colin Loyns, Sheffield (GB)

Correspondence Address: BEYER WEAVER & THOMAS LLP, P.O. BOX 70250, OAKLAND, CA, 94612-0250, US

(21) Appl. No.: 10/516,979

(22) PCT Filed: May 30, 2003
(86) PCT No.: PCT/GB03/02367

§ 371 Date: Aug. 10, 2005

(30) Foreign Application Priority Data
Jun. 13, 2002 (GB) 0213480.7
Jun. 19, 2002 (GB) 0214093.7

(51) Int. Cl. C07C 69/54; B01D 3/00; C07D 225/00

(52) U.S. Cl. 540/450; 203/8

(57) ABSTRACT

A polymerisation inhibitor comprising a non-hindered cyclic hydroxylamine either alone or in combination with an additional inhibitor.

This invention relates to compositions for inhibiting polymerisation of unsaturated monomers, particularly vinyl, α-olefin, acrylic, conjugated diene or other ethylenically unsaturated monomers, and most particularly vinyl aromatic compounds, especially styrene. This invention also relates to a method of inhibiting polymerisation of such monomers.

U.S. Pat. No. 2,965,685 discloses use of N,N-dialkylhydroxylamines to prevent polymerisation of styrene. Various combinations of N,N-dialkylhydroxylamines with other inhibitors have been disclosed.

According to a first aspect of the present invention there is provided a polymerisation inhibitor comprising a non-hindered cyclic hydroxylamine either alone or in combination with an additional inhibitor.

The non-hindered cyclic hydroxylamine is a cyclic hydroxylamine having no alkyl or other alpha substituents adjacent the hydroxylamine group. Preferred compounds have the formula (1). 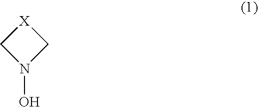
wherein X is a group selected from: (CH₂)ₘY(CH₂)ₙ wherein m and n are each independently an integer from 0 to 5 and Y is a CH₂, or a hetero atom eg O, S or NH and wherein one or more CH₂ is optionally substituted with one or more C₁-C₅ alkyl groups; —(CH₂)ᵣ—CH═CH—(CH₂)ₛ— wherein r and s are independently integers from 0 to 3, optionally substituted with one or more C₁-C₅ alkyl groups.

Preferred examples include: 1-hydroxypiperidine, 4-hydroxymorpholine, 1-hydroxypyrrolidine, 1-hydroxyazetidine, 1-hydroxy-2,5-dihydropyrrole, 1-hydroxyhexamethyleneimine, 1-hydroxyazocan. Partially saturated aromatic bi or tricyclic unhindered hydroxylamines may also be employed, for example, selected from: 1-hydroxy-2,3,4-trihydroquinoline, 9-hydroxycarbozole and 1-hydroxy-2,3-dihydroindole. These compounds may be optionally substituted with one or more C₁-C₅ alkyl groups.

Mixtures of compounds may be employed.

Particularly preferred compounds are selected from: 1-hydroxypiperidine, 4-hydroxymorpholine and mixtures thereof.

The inhibitor in accordance with the first aspect of this invention may be used in combination with one or more co-inhibitors eg nitrophenols such as 2,4-dinitrophenol (DNP) or substituted nitro phenols such as 2-sec-butyl-4,6-dinitrophenol (DNBP). Alternative co-inhibitors may be selected from free radicals (SFR's) such as 4-hydroxy TEMPO, 4-oxo TEMPO, and 4-amino TEMPO, t-alkylcatechols, t-alkylhydroquinones, benzoquinones, p-phenylene diamines and other inhibitors known to those skilled in the art.

The amount of co-inhibitor may be in the range from a trace (eg 1%) to 96%, preferably 40 to 96% by weight of the total amount of inhibitor.

Percentages and other proportions referred to in the specification are by weight unless indicated otherwise. Percentages and proportions may be selected from ranges referred to in the specification to total 100%.

According to a second aspect of the present invention a polymerisation inhibited composition comprises a monomer and an inhibitor in accordance with the first aspect of this invention.

According to a third aspect of this invention a method of inhibiting polymerisation during production, purification, storage or use of a vinyl, α-olefin, acrylic, conjugated diene or other ethylenically unsaturated monomer comprises the step of addition to the monomer of a polymerisation inhibitor in accordance with the first aspect of the present invention.

Unhindered cyclic hydroxylamines in accordance with the present invention have been found to be excellent polymerisation inhibitors, particularly of vinyl aromatic compounds, especially at elevated temperatures. 1-hydroxypiperidine and 4-hydroxymorpholine have been found to be particularly effective inhibitors of styrene polymer formation, both on their own and in combination with 2-sec-butyl-4,6-dinitrophenol (DNBP). Unfavourable premature polymerisation in processing steps such as the production, purification, storage, shipment preparation and use of these monomers or in a mixture of the monomers or a hydrocarbon mixture containing such monomers. Premature polymerisation can cause contamination of the monomer and degradation of the properties of the monomer. A polymer can be deposited in the apparatus. Formation of popcorn polymer is particularly undesirable. The polymerisation inhibitor in accordance with the first aspect of the present invention is effective not only for monomers and mixtures thereof but also for hydrocarbon mixtures and the like containing a small proportion of the monomers.

The invention is further described by means of examples but not in any limitative sense.

Unhindered cyclic hydroxylamines are disclosed in U.S. Pat. No. 2,843,481 (Polaroid) and may be prepared by oxidation of the corresponding amines with aqueous hydrogen peroxide at less than 20° C.
Structures: 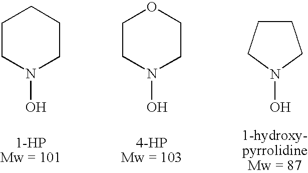
Results
(a) Efficacy

Evaluation of the efficacy of hydroxylamines was carried out using a continuous stirred tank reactor (CSTR). These mimic the reboiler of a styrene distillation column. The styrene has a residence time of approximately two hours inside the reactor.

Two CSTRs were used to gather this data. For any given temperature the same CSTR was used for all experiments at that temperature.

120° C. CSTR—dead volume was 170 ml. With a styrene flow rate of 75 ml/hr the steady state was reached in 4.5 hrs (2 flask volumes). Data gathered after this temperature was averaged to give the steady state polymer level.

110° and 100° C. CSTR—dead volume was 150 ml. With a styrene flow rate of 75 ml/hr the steady state was reached in 4 hrs. Data gathered after this point were averaged to give the steady state polymer level.

Nitrogen sparging to remove oxygen was at a measured rate of 200 ml/minute in all experiments. Aside from the inhibitors under test the only variable was the inherent variation in the rate of thermal initiation of styrene polymerisation.

Hydroxylamines were tested on their own and in combination with DNBP as shown in Table 1 (below). By way of comparison results are also presented for prior art styrene inhibitor mixtures, namely 4-Hydroxy tempo with DNBP, 4-Oxo tempo with DNBP and dihydroxypropylhydroxylamine (DHPHA) with DNBP. At a test temperature of 120° C. the results shown in Table 1 were obtained (polymer results to nearest 50 ppm). Results within 10% of each other have been ranked as equal.

TABLE 1
Results at 120° C. Total inhibitor is 400 ppm

| | | | | Average | |
|---|---|---|---|---|---|
| | | | | Polymer | |
| | | | | at Steady | |
| Component 1 | Wt % | Component 2 | Wt % | State (ppm) | Rank |
| 1-HP | 100 | | | 1850 | 1 |
| DNBP | 90 | 1-HP | 10 | 2500 | 2= |
| DNBP | 90 | 4-HM (100%) | 10 | 2600 | 2= |
| DNBP | 95.5 | 4-Oxo Tempo | 4.5 | 2350 | 2= |
| DNBP | 90 | 4-Hydroxy | 10 | 3200 | 5= |
| | | Tempo | | | |
| DNBP | 100 | | | 3350 | 5= |
| DNBP | 90 | DHPHA | 10 | 3450 | 5= |
| 4-HM (100%) | 100 | | | Failed in | 9 |
| | | | | 3.5 hours | |

Batch tests were also carried out. This was to determine the optimum ratio of DNBP and 4-HM. This was found to be about 7 parts DNBP to about 3 parts 4-HM.

A further continuous test was carried out using this ratio;

TABLE 1a

| | | | | Average Polymer at |
|---|---|---|---|---|
| Component 1 | Wt % | Component 2 | Wt % | Steady State (ppm) |
| DNBP | 70 | 4-HM (100%) | 30 | 1200 |

A further test employed a mixture of 1-Hydroxypiperidine and 4-Hydroxy Tempo. This mixture showed synergy, the results are shown in Table 1b.

TABLE 1b

| | | | | Average Polymer at |
|---|---|---|---|---|
| Component 1 | Wt % | Component 2 | Wt % | Steady State (ppm) |
| 1-HP | 100 | | | 1850 |
| 1-HP | 90 | 4-HT | 10 | 450 |

At 110° C. the results shown in Table 2 were obtained. 4-HM technical grade (65%) showed excellent performance as a single inhibitor at this temperature and therefore the 100% active ingredient was not tested.

TABLE 2
Results at 110° C. Total inhibitor is 250 ppm

| | | | | Average | |
|---|---|---|---|---|---|
| | | | | Polymer | |
| | | | | at Steady | |
| Component 1 | Wt % | Component 2 | Wt % | State (ppm) | Rank |
| 1-HP | 100 | | | 100 | 1 |
| DNBP | 90 | 4-HM (100%) | 10 | 250 | 2 |
| 4-HM (65%) | 100 | | | 700 | 3= |
| DNBP | 90 | 1-HP | 10 | 1100 | 5 |
| DNBP | 90 | 4-Hydroxy | 10 | 1600 | 6 |
| | | Tempo | | | |
| DNBP | 90 | DHPHA | 10 | 1900 | 7 |
| DNBP | 95.5 | 4-Oxo Tempo | 4.5 | 2400 | 9= |
| DNBP | 100 | | | 2400 | 9= |

At 100° C. the results shown in Table 3 were obtained. As before, the results were given to the nearest 50 ppm of polymer and results within 10% of each other were classes as equivalent.

TABLE 3
Results at 100° C. Total 100 ppm inhibitor

| | | | | Average | |
|---|---|---|---|---|---|
| | | | | Polymer | |
| | | | | at Steady | |
| Component 1 | Wt % | Component 2 | Wt % | State (ppm) | Rank |
| 1-HP | 100 | | | 250 | 1 |
| DNBP | 90 | 4-HM (100%) | 10 | 450 | 2 |
| DNBP | 90 | 4-Hydroxy | 10 | 750 | 3 |
| | | Tempo | | | |
| 4-HM (65%) | 100 | | | 1000 | 4 |
| DNBP | 90 | DHPHA | 10 | 1900 | 56 |
| DNBP | 95.5 | 4-Oxo Tempo | 4.5 | 2150 | 67= |
| DNBP | 90 | 1-HP | 10 | 2300 | 67= |
| DNBP | 100 | | | 2750 | 89 |
| DNBP | 100 | | | 2400 | 9= |

### 4-Hydroxymorpholine in 3 Component Systems

In this test N-bis-(1,4-dimethylpentyl)-p-phenylenediamine (PD) was used as a third component. The results are shown in Table 4.

TABLE 4
Three component mixtures

| | Polymer formed | Polymer formed |
|---|---|---|
| | at 120° C. | at 100° C. |
| Test Mixture | (ppm) | (ppm) |
| DNB/PD/DHPHA | 2750 | 250 |
| DNBP/PD/4-HM | 1350 | 100 |

4-Hydroxymorpholine is clearly a superior enhancer of the DNBP/PD system than is DHPHA under our test conditions. It was noted that at 120° C. this three component system is equivalent in performance to the two component DNBP/4-HM system.

1. A method for inhibiting the polymerisation of one or more ethylenically unsaturated monomers selected from the group consisting of:
styrene, α-methylstyrene, styrene sulphonic acid, vinyltoluene, divinylbenzenes, polyvinylbenzenes, alkylated styrene, 2-vinylpyridine, acrylonitrile, methacrylonitrile, methyl acrylate, ethyl acrylate, methyl methacrylate, ethyl methacrylate, acrylic acid, and methacrylic acid;
by adding to the monomers an effective amount sufficient to inhibit polymerisation of a non-hindered cyclic hydroxylamine, alone or in combination with an additional inhibitor.

2. A method as claimed in claim 1, wherein the non-hindered cyclic hydroxylamine has no alkyl or other substituents alpha to the hydroxylamine group.

3. A method as claimed in claim 2, wherein the cyclic hydroxylamine has the formula (1). 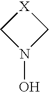
wherein X is a group selected from (CH₂)ₘY(CH₂)ₙ where m and n are each independently an integer from 0 to 5 and Y is a CH₂, or hetero atom such as O, S or NH and wherein one or more CH₂ is optionally substituted with one or more C₁ to C₅ alkyl groups, (CH₂)ᵣ—CH═CH—(CH₂)ₛ where r and s are independently integers from 0 to 3, optionally substituted with one or more C₁ to C₅ alkyl groups.

4. A method as claimed in claim 3 wherein the cyclic hydroxylamine is selected from the group consisting of 1-hydroxypiperidine, 4-hydroxymorpholine, 1-hydroxypyrrolidine, 1-hydroxyazetidine, 1-hydroxy-2,5-dihydropyrrole, 1-hydroxyhexamethylene imine and 1-hydroxyazocan.

5. A method as in claim 2 wherein the cyclic hydroxylamine is selected from the group consisting of 1-hydroxy-2,3,4-trihydroquinoline, 9-hydroxycarbazole and 1-hydroxy-2,3-dihydroindole, optionally substituted with one or more C₁ to C₅ alkyl groups and mixtures thereof.

6. A method as claimed in claim 4, wherein the hydroxylamine is selected from the group consisting of: 1-hydroxypiperidine, 4-hydroxmorpholine and mixtures thereof.

7. A method as claimed in claim 1, wherein the additional inhibitor is selected from the group consisting of phenols, nitrosophenols, nitrophenols, substituted nitrophenols, quinones, stable free radicals and phenylene diamines.

8. A method as claimed in claim 7, wherein the additional inhibitor is selected from the group consisting of: 2,4-dinitrophenol, 4,6-dinitro-o-cresol, 2,6-dinitro-p-cresol, 2-secbutyl-4,6-dintrophenol, tempo, 4-hydroxytempo, 4-oxotempo, 4-aminotempo and 4-methoxytempo.

9. A method as claimed in claim 1, wherein the amount of additional inhibitor in the range from a trace of 96% by weight of the total amount of inhibitor.

10. A method as claimed in claim 9, wherein the amount of additional inhibitor is 40 to 96% by weight of the total amount of inhibitor.

11. A method as claimed in claim 1 wherein the ethylenically unsaturated monomer is styrene.

12. A method as claimed in claim 1, wherein the non-hindered cyclic hydroxylamine is 1-hydroxypiperidine or 4-hydroxymorpholine.

13. A polymerisation inhibitor composition comprising an ethylenically unsaturated monomer selected from the group consisting of: styrene, α-methylstyrene, styrene sulphonic acid, vinyltoluene, divinylbenzenes, polyvinylbenzenes, alkylated styrene, 2-vinylpyridine, acrylonitrile, methacrylonitrile, methyl acrylate, ethyl acrylate, methyl methacrylate, ethyl methacrylate, acrylic acid, and methacrylic acid; and an effective amount sufficient to inhibit polymerisation of a non-hindered cyclic hydroxylamine, alone or in combination with an additional inhibitor.

14. An inhibitor as claimed in claim 13, wherein the non-hindered cyclic hydroxylamine has no alkyl or other substituents alpha to the hydroxylamine group.

15. An inhibitor as claimed in claim 14, wherein the cyclic hydroxylamine has the formula (1). 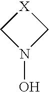
wherein X is a group selected from (CH₂)ₘY(CH₂)ₙ where m and n are each independently an integer from 0 to 5 and Y is a CH₂, or hetero atom such as O, S or NH and wherein one or more CH₂ is optionally substituted with one or more C₁ to C₅ alkyl groups, (CH₂)ᵣ—CH═CH—(CH₂)ₛ where r and s are independently integers from 0 to 3, optionally substituted with one or more C₁ to C₅ alkyl groups.

16. An inhibitor as claimed in claim 15 wherein the cyclic hydroxylamine is selected from the group consisting of: 1-hydroxypiperidine, 4-hydroxymorpholine, 1-hydroxypyrrolidine, 1-hydroxyazetidine, 1-hydroxy-2,5-dihydropyrrole, 1-hydroxyhexamethylene imine and 1-hydroxyazocan.

17. An inhibitor as in claim 16 wherein the cyclic hydroxylamine is selected from the group consisting of 1-hydroxy-2,3,4-trihydroquinoline, 9-hydroxycarbazole and 1-hydroxy-2,3-dihydroindole, optionally substituted with one or more C₁ to C₅ alkyl groups and mixtures thereof.

18. An inhibitor as claimed in claim 16, wherein the hydroxylamine is selected from the group consisting of: 1-hydroxypiperidine, 4-hydroxmorpholine and mixtures thereof.

19. An inhibitor as claimed in claim 13, wherein the additional inhibitor is selected from the group consisting of: phenols, nitrosophenols, nitrophenols, substituted nitrophenols, quinones, stable free radicals and phenylene diamines.

20. An inhibitor as claimed in claim 19, wherein the additional inhibitor is selected from the group consisting of: 2,4-dinitrophenol, 4,6-dinitro-o-cresol, 2,6-dinitro-p-cresol, 2-secbutyl-4,6-dintrophenol, tempo, 4-hydroxytempo, 4-oxotempo, 4-aminotempo and 4-methoxytempo.

21. An inhibitor as claimed in claim 13, wherein the amount of additional inhibitor in the range from a trace of 96% by weight of the total amount of inhibitor.

22. An inhibitor as claimed in claim 21, wherein the amount of additional inhibitor is 40 to 96% by weight of the total amount of inhibitor.

23. An inhibitor as claimed in claim 15, wherein the ethylenically unsaturated monomer is styrene.

24. An inhibitor as claimed in claim 15 wherein the non-hindered cyclic hydroxylamine is 1-hydroxypiperidine or 4-hydroxymorpholine.
